(19) United States
(12) Patent Application Publication
(10) Pub. No.: US 2015/0141520 A1
(43) Pub. Date: May 21, 2015

(54) Stabilized pharmaceutical compositions of fingolimod and process for preparation thereof

(71) Applicants: Chandrasekhar Kandi, Hyderabad (IN); Nagaprasad Vishnubhotla, Hyderabad (IN); Asif Anwar, Hyderabad (IN); Krishnakumar Chegonda, Hyderabad (IN); Sivakumaran Meenakshisunderam, Hyderabad (IN)

(72) Inventors: Chandrasekhar Kandi, Hyderabad (IN); Nagaprasad Vishnubhotla, Hyderabad (IN); Asif Anwar, Hyderabad (IN); Krishnakumar Chegonda, Hyderabad (IN); Sivakumaran Meenakshisunderam, Hyderabad (IN)

(21) Appl. No.: 14/544,040

(22) Filed: Nov. 18, 2014

(30) Foreign Application Priority Data
Nov. 18, 2013 (IN) IN 5289/CHE/2013

(51) Int. Cl. A61K 31/137; A61K 47/14; A61K 47/12; A61K 47/02

(52) U.S. Cl. CPC A61K 31/137; A61K 47/02; A61K 47/14; A61K 47/12
USPC 514/653

(57) ABSTRACT

Stabilized pharmaceutical compositions comprising a S1P receptor modulator as an active agent(s), process of preparation and method of using the same are provided. The present invention also relates to stabilized pharmaceutical compositions comprising fingolimod, or pharmaceutically acceptable salts, esters, hydrates and solvates thereof, process of preparation and method of using the same.

### CROSS REFERENCE TO RELATED APPLICATIONS

This application claims priority from an Indian Patent Application IN 5289/CHE/2013 filed on Nov. 18, 2013.

### FIELD OF THE INVENTION

The present invention relates to stabilized pharmaceutical compositions comprising a SIP receptor modulator as an active agent(s), process of preparation and method of using the same. Particularly the present invention relates to stabilized pharmaceutical compositions comprising fingolimod, or pharmaceutically acceptable salts, esters, hydrates and solvates thereof, process of preparation and method of using the same.

### BACKGROUND OF THE INVENTION

Multiple sclerosis (MS) also known as disseminated sclerosis or encephalomyelitis disseminata is a disease in which the nerves of the central nervous system (brain and spinal cord) degenerate. Myelin provides a covering or insulation for nerves, improves the conduction of impulses along the nerves and also is important for maintaining the health of the nerves. In multiple sclerosis, inflammation causes the myelin to disappear. Consequently, the electrical impulses that travel along the nerves decelerate. In addition, the nerves themselves are damaged. As more and more nerves are affected, patient suffer from a range of symptoms which affect their health related quality of life such as pain, muscle spasticity and spasm, bladder problems and sleep disturbance.

Although there is no known cure for multiple sclerosis, there are few effective treatments (marketed medications) for the symptoms of multiple sclerosis that tend to improve function after an attack and prevent new attacks. Nevertheless, it is reported that only a minority of people can benefit from current drugs and associated adverse side effects significantly limit use of these drugs. Despite the introduction of some different medications for the treatment of multiple sclerosis over years, there is a very clear need for alternative new treatments for MS.

Fingolimod, which is also referred to as “FTY720” and the first-in-class orally available drug, is a synthetic imitation of myriocin; a metabolic product of the fungus Isaria sinclairii. Fingolimod is a sphingosine-1 phosphate (SIP) agonist, having immunosuppressive activity. The IUPAC name of fingolimod is 2-amino-2-(2-[4-octylphenyl] ethyl)-1,3-propane diol and its chemical structure is shown below:

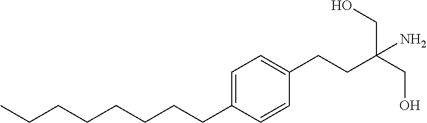 [SMILES: CCCCCCCCc1ccc(CCC(N)(CO)CO)cc1]

Fingolimod is currently marketed as Gilenya® immediate release capsule for the treatment of multiple sclerosis. This formulation contains 0.5 mg equivalent of fingolimod base in the form of the hydrochloride salt. Each GILENYA 0.5 mg capsule contains the following inactive ingredients: gelatin, magnesium stearate, mannitol, titanium dioxide, yellow iron oxide.

The synthesis of fingolimod is described, for example, in the European patent application no. EP 0627406. U.S. Pat. No. 5,604,229 specifically discloses the product Fingolimod and other related compounds. It has been found to be useful in the treatment or prevention of various autoimmune conditions, including multiple sclerosis.

US patent publication 2010/0040678 discloses rapidly disintegrating dosage forms of S1P agonists including FTY720 (fingolimod). The compositions comprise a coating, wherein the coating comprises one or more polymer resins and one or more metal oxides.

U.S. Pat. No. 8,324,283 patent, US 2006/0275357 patent publication, US 2009/0203798 patent publication, US 2011/0105620 patent publication and US 2013/0108675 patent publication disclose pharmaceutical compositions of S1P agonists comprising a sugar alcohol, such as mannitol. The sugar alcohol may act as a diluent, carrier, filler or bulking agent and may suitably be mannitol, maltitol, inositol, xylitol and/or lactitol. It is taught in the abovesaid patents that these compositions show a high level of content uniformity as well as high stability. The composition may be in a form of a powder, granule, pellet or a tablet. In examples of preferred embodiment, fingolimod hydrochloride is mixed with mannitol and lubricant and, optionally with a binder such as HPC (hydroxy propyl cellulose) or HPMC (hydroxy propyl methylcellulose), milled and/or granulated. The said patents appear to cover the marketed formulation of fingolimod capsules.

US 2013/0034603 patent publication discloses a process for preparing a pharmaceutical composition of fingolimod comprising: (i) obtaining a intimate admixture comprising fingolimod or a pharmaceutically acceptable salt thereof, and at least one surfactant (wetting agent) (ii) optionally combining the intimate admixture from step (i) with one or more excipients.

PCT publication WO 2009/048993 discloses dosage forms containing S1P modulators (such as e.g. fingolimod) and one or more excipients selected from fillers, binders, disintegrants, lubricants, flow regulators, matrix formers, plasticizers, flavoring agents and sweeteners. As per the said publication compounds with amine substitution are not easy to formulate in a solid oral formulation; only a limited number of excipients are potentially feasible with such amino diols. In particular, reducing sugars are not considered suitable due to danger of Maillard reaction with the amino-group. Thus, according to the teachings of the abovesaid publication, the only suitable fillers like those of lactose, lactose monohydrate, maize starch, mannitol, xylitol, sorbitol, sucrose, microcrystalline cellulose, dibasic calcium phosphate, maltodextrin and gelatin provided stable blends with the aminopropane-1-3-diol based SIP receptor modulator.

PCT publication WO 2010/055028 discloses crystalline forms and hydrates of fingolimod hydrochloride and pharmaceutical formulations thereof. The solid pharmaceutical formulations comprise the crystalline fingolimod hydrochloride and a sugar alcohol. The sugar alcohol can be, e.g. mannitol, maltitol, inositol, xylitol or lactitol. PCT publication WO2011/131368 discloses a method of preparing an intermediate containing fingolimod, a method of preparing granules containing fingolimod, a method of preparing an oral dosage form containing fingolimod and accordingly intermediates, granules and oral dosage forms obtainable by that method.

PCT publication WO2013/091704 discloses compositions comprising ingolimod and/or a salt or an ester thereof, and calcium lactate pentahydrate. The said publication teaches that calcium lactate pentahydrate may form stable compositions with Fingolimod, and is superior to many other suggested fillers. The compositions of the invention exhibit good handling properties, e.g. flowability, content uniformity etc. for making powders and-or granulates both for direct oral administration and-or for tableting. Furthermore, calcium lactate pentahydrate efficiently masks the unpleasant taste of Fingolimod without providing a sweet taste as, e.g., mannitol and similar sugar alcohols may do. PCT publication WO2014141298 discloses stable pharmaceutical composition of fingolimod comprising fingolimod or its pharmaceutically acceptable salt thereof, polysaccharide and at least one pharmaceutically acceptable excipient.

Several disclosures in the prior art mention that fingolimod being an amino substituted product has content uniformity issues, and stability issues as a result due to Maillard reaction of amino groups with sugar alcohol. Further, fingolimod possess properties that can cause processing problems whilst preparing pharmaceutical formulations. In particular, it has been found that fingolimod particles have a strong tendency to stick to surfaces and to each other. Further, fingolimod can react with certain excipients to produce degradation products in the final formulation. It is also known in the prior art that magnesium stearate is a hydrophobic lubricant and can result in poor disintegration and dissolution of the active ingredient. Thus, due to stability, content uniformity, processing issues and drawbacks of magnesium stearate as a lubricant in the prior art, there is still a need to develop stabilized formulations comprising fingolimod which at the same time address the issues present in the prior art as well as result in a formulation which are more robust, economical, have good storage stability, are less time consuming and have comparable dissolution and bioavailability with respect to the marketed product Gilenya® (Fingolimod hydrochloride capsules) due to use of conventional lubricants other than magnesium stearate.

### SUMMARY OF THE INVENTION

An aspect of the present invention provides stable oral pharmaceutical compositions comprising S1P receptor modulator as an active agent(s) or pharmaceutically acceptable salts, esters, hydrates and solvates thereof, atleast one excipient(s) which is not a sugar alcohol, optionally with one or more other pharmaceutically acceptable excipient(s).

An aspect of the present invention provides stable oral pharmaceutical compositions comprising fingolimod as an active agent(s) or pharmaceutically acceptable salts, esters, hydrates and solvates thereof, atleast one excipient(s) which is not a sugar alcohol, optionally with one or more other pharmaceutically acceptable excipient(s).

An aspect of the present invention provides stable oral pharmaceutical compositions comprising fingolimod as an active agent(s) or pharmaceutically acceptable salts, esters, hydrates and solvates thereof, atleast one saccharide(s), optionally with one or more pharmaceutically acceptable excipient(s).

Another aspect of the present invention provides stable oral pharmaceutical compositions comprising fingolimod as an active agent(s) or pharmaceutically acceptable salts, esters, hydrates and solvates thereof, atleast one saccharide(s) selected from group comprising monosaccharides, disaccharides, oligosaccharides and polysaccharides, optionally with one or more pharmaceutically acceptable excipients.

In an aspect, the present invention provides process for the preparation of stable oral pharmaceutical compositions, wherein the process comprises of the following steps:

(i) treating fingolimod with atleast one excipient which is not a sugar alcohol,

(ii) optionally adding one or more other pharmaceutically acceptable excipients, and

(iii) formulating the material of step (i) and (ii) into a suitable dosage form.

In an aspect, the present invention provides process for the preparation of stable oral pharmaceutical compositions, wherein the process comprises of the following steps:

(i) treating fingolimod with one or more saccharide(s),

(ii) optionally adding one or more pharmaceutically acceptable excipients, and

(iii) formulating the material of step (i) and (ii) into a suitable dosage form.

An aspect of the present invention relates to method of using such compositions for treatment of patients with autoimmune disorders particularly with relapsing forms of multiple sclerosis to reduce the frequency of clinical exacerbations and to delay the accumulation of physical disability. An aspect of the present invention relates to method of treating a patient suffering from autoimmune disorders particularly with relapsing forms of multiple sclerosis to reduce the frequency of clinical exacerbations and to delay the accumulation of physical disability.

### DETAILED DESCRIPTION OF THE INVENTION

The term ‘stable’ refers to formulations that substantially retain the label amount of the therapeutically active ingredient during storage for commercially relevant times, and the drug-related impurity contents in the formulations remain within acceptable limits. Further, the term ‘stable’ also optionally refers to formulations that contain polymorphically stable active ingredient. The phrase “substantially pure polymorphic form of fingolimod”, unless otherwise specified is to be understood as a substance free of other polymorphic and/or pseudopolymorphic forms at amounts detectable with typical analytical methods such as X-ray powder diffraction and/or solid state infrared absorption, i.e. containing less than 10% of other polymorphic and/or pseudopolymorphic forms.

The term ‘pharmaceutically acceptable’ as used herein, refers to materials that are suitable for use in contact with the tissues of humans and lower animals without undue toxicity, irritation, allergic response, and the like, in keeping with a reasonable benefit-risk ratio, and effective for their intended use.

The term “fingolimod” unless indicated otherwise, refers to fingolimod in its free base form, or as a pharmaceutically acceptable salt, or esters, hydrates and solvates thereof. Preferably fingolimod is in the form of a pharmaceutically acceptable acid addition salt, more preferably, in the form of its hydrochloride salt. According to the present invention, 90% of particles with particle size less than about 100 μm, and/or surface area less that about 5 m²/gm are useful. Particularly according to the present invention, 90% of particles with particle size less than about 50 μm and/or surface area less that about 5 m²/gm are useful.

The term “composition” or “pharmaceutical composition” or “dosage form” as used herein synonymously include solid dosage forms such as granules, multiunit particulate systems (MUPS), pellets, spheres, tablets, capsules, mini-tablets, beads, particles and the like; and liquid dosage forms such as solutions, suspensions, emulsions, colloids and the like, meant for oral administration.

As used in this specification, the singular forms “a”, “an”, and “the” include plural references unless the context clearly dictates otherwise. Thus for example, a reference to “a process” includes one or more process, and/or steps of the type described herein and/or which will become apparent to those persons skilled in the art upon reading this disclosure and so forth.

The present invention provides stable oral pharmaceutical compositions comprising S1P receptor modulator as an active agent(s) or pharmaceutically acceptable salts, esters, hydrates and solvates thereof, at least one excipient(s) which is not a sugar alcohol, optionally with one or more other pharmaceutically acceptable excipient(s).

In an embodiment, the present invention provides stable oral pharmaceutical compositions comprising fingolimod as an active agent(s) or pharmaceutically acceptable salts, esters, hydrates and solvates thereof, atleast one excipient(s) which is not a sugar alcohol, optionally with one or more other pharmaceutically acceptable excipient(s).

In an embodiment, the atleast one excipient(s) which is not a sugar alcohol, may be selected from but not limited to a group comprising saccharides, celluloses, cellulose derivatives, dicalcium phosphate, tribasic calcium phosphate, dihydrogen sodium phosphate, carbonates or bicarbonates or oxides or hydroxides of metals such as alkali or alkaline earth metals, alginates, carboxyalkyl celluloses, gums, amido group containing polymers, amino group containing polymers, sugars, hydrophobic compounds such as waxes, and the like, and mixtures thereof.

In yet another embodiment, the present invention provides stable oral pharmaceutical compositions comprising fingolimod as an active agent(s) or pharmaceutically acceptable salts, esters, hydrates and solvates thereof, atleast one saccharide(s), optionally with one or more pharmaceutically acceptable excipients.

In an embodiment, the present invention provides stable oral pharmaceutical compositions comprising fingolimod as an active agent(s) or pharmaceutically acceptable salts, esters, hydrates and solvates thereof, atleast one saccharide(s) selected from group comprising monosaccharide(s), disaccharide(s), oligosaccharide(s) and polysaccharide(s), optionally with one or more pharmaceutically acceptable excipients.

In another embodiment, the present invention provides stable oral pharmaceutical compositions comprising fingolimod as an active agent(s) or pharmaceutically acceptable salts, esters, hydrates and solvates thereof, at least one oligosaccharide(s) optionally with one or more pharmaceutically acceptable excipients.

In an embodiment, the monosaccharide(s) useful in the present invention are selected from but not limited to sorbitol, dextrose, fructose, maltose and xylitol; disaccharide(s) selected from but not limited to glucose, galactose and mannitol; oligosaccharide(s) selected from but not limited to dextrates, cyclodextrins and maltodextrins (e.g. Glucidex®); polysaccharide(s) selected from but not limited to xanthan gum, guar gum, gum arabic, carrageenan gum, karaya gum, locust bean gum, acacia gum, tragacanth gum, agar, pectin, furcellaran, zein, casein, starch and the like thereof used either alone or in combination thereof. In a preferred embodiment, the saccharide is an oligosaccharide more preferably maltodextrin.

In an embodiment, the known impurities (impurity 1 & 2) are not more than 1% under 25°/60%RH—real time storage condition (6 months) or 40°/75%RH—accelerated storage conditions (3 months). “Impurity 1” is herein referred to as 2-acetamido-2-(4-octanoylphenethyl)propane-1,3-diyldiacetate and “Impurity 2” is herein referred to as 2-acetamido-2-(4-octylphenethyl)propane-1,3-diyldiacetate. In another embodiment, the maximum individual unknown impurities are not more than 1% under 25°/60%RH—real time storage condition (6 months) or 40°/75%RH—accelerated storage conditions (3 months). In yet another embodiment, total impurities are not more than 5% under 25°/60%RH—real time storage condition (6 months) or 40°/75%RH—accelerated storage conditions (3 months).

In one of the embodiments, the present invention provides stable oral pharmaceutical compositions comprising fingolimod as an active agent(s) or pharmaceutically acceptable salts, esters, hydrates and solvates thereof from about 0.1% w/w to about 99% w/w of the composition, maltodextrin from about 0.1% to about 99% w/w of the composition, optionally with one or more pharmaceutically acceptable excipients, from about 0.9% to about 97% w/w of the composition.

In an embodiment, the present invention provides stable oral pharmaceutical compositions comprising fingolimod as an active agent(s) or pharmaceutically acceptable salts, esters, hydrates and solvates thereof from about 0.1% w/w to about 99% w/w of the composition, maltodextrin from about 0.1% to about 99% w/w of the composition, optionally with one or more pharmaceutically acceptable excipients from about 0.9% to about 97% w/w of the composition and wherein the composition further comprises not more than about 1% w/w of 2-acetamido-2-(4-octanoylphenethyl)propane-1,3-diyldiacetate and/or not more than about 1% w/w of 2-acetamido-2-(4-octylphenethyl)propane-1,3-diyldiacetate, when stored at a temperature of about 40° C. and relative humidity of about 75% for 3 months or more.

In a preferred embodiment, the present invention provides 0.001% to about 0.5% w/w of 2-acetamido-2-(4-octanoylphenethyl)propane-1,3-diyldiacetate and/or 2-acetamido-2-(4-octylphenethyl)propane-1,3-diyldiacetate. In a more preferred embodiment, the present invention provides less than about 0.05% w/w of 2-acetamido-2-(4-octanoylphenethyl)propane-1,3-diyldiacetate and/or less than about 0.02% w/w of 2-acetamido-2-(4-octylphenethyl)propane-1,3-diyldiacetate.

In an embodiment the present invention provides stable oral pharmaceutical compositions, wherein the ratio of active agent(s) to maltodextrin is from about 0.1:100 to about 100:0.1. Preferably the ratio of active agent(s) to maltodextrin is from about 0.1:90 to about 90:0.1.

In an embodiment, the present invention provides process for the preparation of stable oral pharmaceutical compositions, wherein the process comprises of the following steps:

(i) treating fingolimod with atleast one excipient(s) which is not a sugar alcohol,

(ii) optionally adding one or more other pharmaceutically acceptable excipients, and

(iii) formulating the material of step (i) and (ii) into a suitable dosage form

In another embodiment is provided process for the preparation of stable oral pharmaceutical compositions, wherein the process comprises of the following steps:

(i) treating fingolimod with saccharide(s),

(ii) optionally adding one or more pharmaceutically acceptable excipients, and

(iii) formulating the material of step (i) and (ii) into a suitable dosage form

In one of the embodiments is provided process for the preparation of stable oral pharmaceutical compositions, wherein the process comprises of the following steps:

(i) treating fingolimod with maltodextrin,

(ii) optionally adding one or more pharmaceutically acceptable excipients, and

(iii) compressing the granules, to form a tablet or filling into capsules.

As used herein, unless indicated otherwise, references to total weight of the pharmaceutical composition refers to the total weight of the active agent(s) and pharmaceutically acceptable excipient(s), and excludes any capsule shell.

“Pharmaceutically acceptable excipient(s)” are components added to active agent pharmaceutical formulation other than the active ingredient fingolimod. Excipients may be added to facilitate manufacture, enhance stability, enhance product characteristics, enhance bioavailability, enhance patient acceptability, etc. Pharmaceutically acceptable excipient(s) includes, but not limited to, one or more of diluents/fillers, binders, disintegrants, lubricants, glidants, compression aids, colors, sweeteners, preservatives, surfactants, suspending agents, dispersing agents, film formers, flavors, printing inks, etc.

Binders hold the ingredients in the composition together. Exemplary binders are selected from, but not limited to, cellulose and its derivatives including, ethyl cellulose, hydroxypropyl cellulose, hydroxypropyl methylcellulose, methylcellulose and hydroxyethyl cellulose, carboxymethyl cellulose; gelatin, liquid glucose; starch and its derivatives; hydrocolloids; sugars; polyvinyl pyrrolidone, sodium alginate, acacia, alginic acid, tragacanth, used either alone or combinations comprising one or more of the foregoing binders. The binder may be used in the range of 1-10% by weight of the composition.

Diluents increase the bulk of the composition. Diluents according to the present invention are selected from, but not limited to, sugars such as lactose, sucrose, dextrose; sugar alcohols such as mannitol, sorbitol, xylitol, lactitol; starlac, starch, modified starches, dibasic calcium phosphate, tribasic calcium phosphate, calcium carbonate, calcium sulfate, powdered cellulose, microcrystalline cellulose, silicified microcrystalline cellulose, magnesium carbonate, magnesium oxide and the like used either alone or in combinations thereof. The diluent may be used in the range of 50-98% by weight of the composition.

Disintegrants according to the present invention are selected from, but not limited to, cellulose and its derivatives including low-substituted hydroxypropyl cellulose; cross-linked polyvinylpyrrolidone; cross-linked sodium carboxymethylcellulose, sodium carboxymethylcellulose, microcrystalline cellulose; sodium starch glycolate; ion-exchange resins; starch and modified starches including pregelatinized starch; formalin-casein; used either alone or in combinations comprising one or more of the foregoing water swellable substances.

Lubricants and glidants aids in the processing of powder materials. Exemplary lubricants are selected from, but not limited to, calcium stearate, magnesium stearate, glycerol behenate, mineral oil, polyethylene glycol, sodium stearyl fumarate, stearic acid, talc, vegetable oil, and zinc stearate, used either alone or in combinations comprising one or more of the foregoing lubricants. Exemplary glidants include, but not limited to, talc, silicon dioxide, cornstarch and the like used either alone or in combination thereof.

In another embodiment, the ratio of the fingolimod or pharmaceutically acceptable salts, esters, hydrates and solvates thereof to lubricants is preferably about 0.1% to about 1% w/w, more preferably about 0.2% to about 0.9% w/w.

Surfactants are compounds which are capable of improving the wetting of the drug and/or enhancing the dissolution. The surfactants can be selected from hydrophilic surfactants or lipophilic surfactants or mixtures thereof. The surfactants can be anionic, nonionic, cationic, and zwitterionic surfactants. Surfactants according to the present invention are selected from, but not limited to, polyoxyethylene alkylaryl ethers such as polyoxyethylene lauryl ether, polyoxyethylene cetyl ether, polyoxyethylene stearyl ether; polyethylene glycol (PEG) fatty acid esters such as PEG monolaurate, PEG dilaurate, PEG distearate, PEG dioleate; polyoxyethylene sorbitan fatty acid ester such as polysorbate 40, polysorbate 60, polysorbate 80; sorbitan fatty acid mono esters such as sorbitan monolaurate, sorbitan monooleate, sorbitan sesquioleate, sorbitan trioleate, polyoxyethylene castor oil derivates such as polyoxyl castor oil, polyoxyl hydrogenated castor oil, sodium lauryl sulphate and the like used either alone or in combination thereof. “Suitable solvent” according to the present invention can be any solvent in which the binder is soluble or dispersible and is selected from isopropyl alcohol, ethanol, water, acetone, methylene chloride and the like or mixtures thereof.

It must be appreciated that the pharmaceutical compositions of the present invention can include all the dosage forms known to a person skilled in art, viz. oral formulations such as single unit dosage forms in the form of tablets, minitablets filled in capsules and the like; beads, pellets presented in a sachet, capsule or tablet capsules such as soft and hard gelatin; lozenges or sachets; granulates, microparticles, multiparticulates, powder and the like.

In an embodiment, the compositions of the present invention may additionally comprise of a colorant in order to produce a desirable color. Colors known to be ‘FD&C’ certified may be used to provide coloring to the product and are within the purview of the present invention. Suitable colorants include natural colorants i.e., pigments and dyes obtained from mineral, plant, and animal sources. Examples of natural colorants include red ferric oxide, yellow ferric oxide, annattenes, alizarin, indigo, rutin, quercetin, and the like. Synthetic colorants may also be used, which is typically an FD&C or D&C dye, e.g., an approved dye selected from the so-called ‘coal-tar’ dyes, such as a nitroso dye, a nitro dye, an azo dye, an oxazine, a thiazine, a pyrazolone, a xanthene, an indigoid, an anthraquinone, an acridine, a rosaniline, a phthalein, a quinoline, or a ‘lake’ thereof, i.e. an aluminum or calcium salt thereof. Particularly preferred colorants are food colorants in the ‘GRAS’ (Generally Regarded as Safe) category.

In an embodiment, the tablet compositions of the present invention may be film coated. A film forming agent may provide smooth film-forming coating suspensions and enhance the rheological mechanical strength properties of film coating gel matrices. Film forming agents include, for example, polyvinylpyrrolidone, natural gums, starches, and cellulosic polymers. A cellulosic polymer may include a molecule comprising at least one cellulose polymer or derivative modified with small amounts of propylene glycol ether groups attached to the cellulose anhydroglucose chain affording binding properties that enhance the reinforcing film properties of film applications. Examples of cellulosic polymers include, but are not limited to, hydroxypropyl methyl cellulose (“HPMC”), carboxymethyl cellulose (“CMC”) or salts thereof, hydroxypropyl cellulose (“HPC”), methylcellulose (“MC”), hydroxyethyl cellulose (“HEC”), and the like. In addition, cellulosic polymers may be characterized as ionic or non-ionic. Ionic cellulosic polymers include, for example, sodium CMC. Non-ionic cellulosic polymers include, for example, HPMC, HPC, HEC, and MC. Varieties of commercially available cellulosic polymers exist and may include, for example, Spectracel® HPMC compositions (available from Sensient Technologies). Further, other commercially available coating materials are available marketed under the brand name Opadry® for example Opadry II Gray which contains: lactose monohydrate NF, hypromellose type 2910 USP, titanium dioxide USP, triacetin USP, and iron oxide black JPE; Opadry II Pink which contains: hypromellose type 2910 USP, titanium dioxide USP, lactose monohydrate NF, polyethylene glycol 3350 NF, triacetin USP, and FD&C Red #40; Opadry II Blue which contains: hypromellose type 2910 USP, lactose monohydrate NF, FD&C Blue #1, polyethylene glycol 3350 NF, FD&C Blue #2, titanium dioxide USP, triacetin USP, and D&C Yellow #10; Opadry II Yellow which contains: hypromellose type 2910 USP, lactose monohydrate NF, titanium dioxide USP, iron oxide yellow NF, polyethylene glycol 3350 NF, and triacetin USP; Opadry II Purple which contains: hypromellose type 2910 USP, lactose monohydrate NF, titanium dioxide USP, D&C Red #27, polyethylene glycol 3350 NF, triacetin USP, and FD&C Blue #1 and the like.

In another embodiment, the present invention provides method of using such compositions for treatment of patients with autoimmune disorders particularly with relapsing forms of multiple sclerosis to reduce the frequency of clinical exacerbations and to delay the accumulation of physical disability.

The compositions of the present invention can be packed into suitable containers such as bottles, blisters or pouch. Further, the packages may optionally contain a dessicant or an antioxidant or oxygen absorbant or combinations thereof.

The following examples serve to illustrate the embodiments of the present invention. However, they do not intend to limit the scope of the invention. It is obvious to those skilled in the art to find out the composition for other dosage forms and substitute the equivalent excipients as described in this specification or with the one known to the industry.

### EXAMPLE 1

| S. No. | Ingredients | % w/w |
|---|---|---|
| | Intragranular | |
| 1 | Fingolimod hydrochloride | 1.17 |
| 2 | Microcrystalline cellulose | 46.45 |
| 3 | Lactose monohydrate | 39.50 |
| 4 | Purified water* | q.s. |
| | Extragranular | |
| 5 | Hypromellose | 8.00 |
| 6 | Pregelatinized starch | 3.00 |
| 7 | Magnesium stearate | 1.88 |

*q.s.: Lost in processing

### Manufacturing Process:

i) Fingolimod hydrochloride and microcrystalline cellulose and lactose monohydrate were sifted together.

ii) The blend of step (i) was granulated with water as and was dried.

iii) The granules obtained in step (ii) were blended with the extragranular hypromellose and pregelatinized starch.

iv) The granules of step (iii) were lubricated with magnesium stearate.

v) The lubricated granules of step (iv) was filled into capsules.

### EXAMPLE 2

| | Ingredients | |
|---|---|---|
| S. No. | Intragranular | % w/w |
| 1 | Fingolimod hydrochloride | 1.17 |
| 2 | Dihydrogen sodium phosphate | 33.33 |
| 3 | Tribasic calcium phosphate | 61.50 |
| 4 | Silicon dioxide | 2.00 |
| 5 | Calcium stearate | 2.00 |

### Manufacturing Process:

i) Fingolimod hydrochloride, dihydrogen sodium phosphate, tribasic calcium phosphate and silicon dioxide were sifted together.

ii) The blend of step (i) was slugged/compacted and milled.

iii) The granules obtained in step (ii) were lubricated with calcium stearate.

iv) The lubricated granules of step (iii) was filled into capsules.

### EXAMPLE 3

| S. No. | Ingredients | % w/w |
|---|---|---|
| 1 | Fingolimod hydrochloride | 1.17 |
| 2 | Sodium carboxymethyl cellulose | 44.80 |
| 3 | Dibasic calcium phosphate | 42.30 |
| 4 | Sodium lauryl sulfate | 1.30 |
| 5 | Pregelatinized starch | 8.63 |
| 6 | Glyceryl behenate | 1.80 |

Coating

| | | |
|---|---|---|
| 7 | Opadry II Pink | 3.00 |
| 8 | Purified water* | q.s. |

*q.s.: Lost in processing

### Manufacturing Process:

i) Fingolimod hydrochloride, sodium carboxymethyl cellulose, dibasic calcium phosphate, sodium lauryl sulfate, and pregelatinized starch were sifted together.

ii) The blend of step (i) was slugged/compacted and milled.

iii) The granules obtained in step (ii) were lubricated with glyceryl behenate.

iv) The lubricated granules of step (iii) was compressed to tablets.

### Coating of Tablets:

v) The tablets of step (iv) were then film coated with Opadry II Pink.

### EXAMPLE 4

| S. No. | Ingredients | % w/w |
|---|---|---|
| 1 | Fingolimod hydrochloride | 1.17 |
| 2 | Crospovidone | 18.00 |
| 3 | Lactose monohydrate | 70.00 |
| 4 | Sodium lauryl sulfate | 1.61 |
| 5 | Pregelatinized starch | 8.00 |
| 6 | Zinc stearate | 1.22 |

### Manufacturing Process:

i) Fingolimod hydrochloride, crospovidone, lactose monohydrate, sodium lauryl sulfate, and pregelatinized starch were sifted together.

ii) The blend of step (i) was slugged/compacted and milled.

iii) The granules obtained in step (ii) were lubricated with zinc stearate.

iv) The lubricated granules of step (iii) was compressed to tablets.

### EXAMPLE 5

| S. No. | Ingredients | % w/w |
|---|---|---|
| 1 | Fingolimod hydrochloride | 1.17 |
| 2 | Sodium bicarbonate | 24.93 |
| 3 | Tribasic calcium phosphate | 68.50 |
| 4 | Polyvinyl pyrrolidone | 3.6 |
| 5 | Purified water | q.s. |
| 6 | Stearic acid | 1.8 |

Coating

| | | |
|---|---|---|
| 7 | Opadry II Yellow | 3.00 |
| 8 | Purified water* | q.s. |

*q.s.: Lost in processing

### Manufacturing Process:

(i) Fingolimod hydrochloride, sodium bicarbonate, tribasic calcium phosphate were sifted and blended together to form a dry blend,

(ii) Binder solution was prepared by dissolving polyvinyl pyrrolidone in purified water,

(iii) The blend of step (i) was granulated with binder solution of step (ii), using rapid mixer granulator,

(iv) The granules of step (iii) were dried and milled to get the desired granules of fingolimod hydrochloride,

(v) The blend of step (iv) was lubricated with stearic acid,

(vi) The lubricated granules of step (v) were finally compressed into tablets.

### Coating of Tablets:

vii) The tablets of step (vi) were then film coated with Opadry II Yellow.

### EXAMPLE 6

| | Ingredients | |
|---|---|---|
| S. No. | Intragranular | % w/w |
| 1 | Fingolimod hydrochloride | 1.17 |
| 2 | Glucidex ® (maltodextrin) | 94.83 |
| 3 | Silicon dioxide | 2.00 |
| 4 | Sodium stearyl fumarate | 2.00 |

### Manufacturing Process:

i) Maltodextrin was milled and passed through sieve of mesh size 60.

ii) Fingolimod hydrochloride and silicon dioxide were sifted together.

iii) The material of step (ii) was blended with half the quantity of maltodextrin and then passed through sieve of mesh size 60.

iv) The material of step (iii) was co-sifted with the remaining quantity of maltodextrin and then passed through sieve of mesh size 60.

v) Sodium stearyl fumarate was sifted through sieve of mesh size 80.

vi) The granules obtained in step (iv) were lubricated with sodium stearyl fumarate of step (v).

vii) The lubricated blend of step (vi) was filled into capsules.

| | Example 6 | GILENYA ® |
|---|---|---|
| Time | Percent drug release | Percent drug release |
| 0 | 0 | 0 |
| 10 | 79 | 96 |
| 15 | 88 | 99 |
| 20 | 91 | 99 |
| 30 | 93 | 100 |
| 45 | 94 | 100 |
| 60 | 95 | 100 |

### Stability Data:

The fingolimod hydrochloride capsules of example 3 and example 7 were packed in Cold Form (3 Layer) blisters and PVC/PVDC blisters and subjected to stability testing at 40° C.±2° C./75% RH±5% RH for 3 months.

| | Example 6 | |
|---|---|---|
| Impurity | Cold Form (3 layer) Blister | PVC/PVDC Blister |

| | |
|---|---|
| Impurity - 1 | Less than 0.05% |
| Impurity - 2 | Less than 0.02% |

### EXAMPLE 7

| | Ingredients | |
|---|---|---|
| S. No. | Intragranular | % w/w |
| 1 | Fingolimod hydrochloride | 1.17 |
| 2 | Glucidex ® (maltodextrin) | 94.83 |
| 3 | Talc | 2.00 |
| 4 | Sodium stearyl fumarate | 2.00 |

### Manufacturing Process:

i) Maltodextrin was milled and passed through sieve of mesh size 60.

ii) Fingolimod hydrochloride and Talc were sifted together though sieve of mesh size 80.

iii) The material of step (ii) was blended with half the quantity of maltodextrin and then passed through sieve of mesh size 60.

iv) The material of step (iii) was co-sifted with the remaining quantity of maltodextrin and then passed through sieve of mesh size 60.

v) Sodium stearyl fumarate was sifted through sieve of mesh size 80.

vi) The granules obtained in step (iv) were lubricated with sodium stearyl fumarate of step (v).

vii) The lubricated blend of step (vi) was filled into capsules.

### Dissolution Data:

Dissolution profiles of the fingolimod hydrochloride capsules of example 3; example 7 and Reference product (GILENYA®) were carried with USP apparatus I at 100 rpm using 500 ml in 0.1 N HCl with 0.2% SLS.

| | Example 7 | GILENYA ® |
|---|---|---|
| Time | Percent drug release | Percent drug release |
| 0 | 0 | 0 |
| 10 | 66 | 96 |
| 15 | 81 | 99 |
| 20 | 88 | 99 |
| 30 | 95 | 100 |
| 45 | 101 | 100 |
| 60 | 103 | 100 |

### Stability Data:

The fingolimod hydrochloride capsules of example 3 and example 7 were packed in Cold Form (3 Layer) blisters and PVC/PVDC blisters and subjected to stability testing at 40° C.±2° C./75% RH±5% RH for 3 months.

| | Example 7 | |
|---|---|---|
| Impurity | Cold Form (3 layer) Blister | PVC/PVDC Blister |

| | |
|---|---|
| Impurity - 1 | Less than 0.05% |
| Impurity - 2 | Less than 0.02% |

We claim:

1. Stable oral pharmaceutical compositions comprising fingolimod as an active agent(s) or pharmaceutically acceptable salts, esters, hydrates and solvates thereof, atleast one excipient(s) which is not a sugar alcohol, optionally with one or more other pharmaceutically acceptable excipient(s).

2. The compositions according to claim 1, wherein the atleast one excipient(s) which is not a sugar alcohol, is selected from a group comprising saccharides, celluloses, cellulose derivatives, dicalcium phosphate, tribasic calcium phosphate, dihydrogen sodium phosphate, carbonates or bicarbonates or oxides or hydroxides of metals, alginates, carboxyalkyl celluloses, gums, amido group containing polymers, amino group containing polymers, sugars, hydrophobic compounds and mixtures thereof.

3. The compositions according to claim 2, wherein saccharide(s) is selected from group comprising monosaccharide(s), disaccharide(s), oligosaccharide(s) and polysaccharide(s), optionally with one or more pharmaceutically acceptable excipients.

4. The compositions according to claim 3, wherein the monosaccharide(s) are selected from sorbitol, dextrose, fructose, maltose and xylitol; disaccharide(s) selected from glucose, galactose and mannitol; oligosaccharide(s) selected from dextrins, dextrates, cyclodextrins and maltodextrins; polysaccharide(s) selected from xanthan gum, guar gum, gum arabic, carrageenan gum, karaya gum, locust bean gum, acacia gum, tragacanth gum, agar, pectin, furcellaran, zein, casein, starch used either alone or in combination thereof.

5. The compositions according to claims 1 to 4, comprising fingolimod as an active agent(s) or pharmaceutically acceptable salts, esters, hydrates and solvates thereof from about 0.1% w/w to about 99% w/w of the composition, maltodextrin from about 0.1% to about 99% w/w of the composition, optionally with one or more pharmaceutically acceptable excipients, from about 0.9% to about 97% w/w of the composition.

6. The compositions according to claim 5, comprising fingolimod as an active agent(s) or pharmaceutically acceptable salts, esters, hydrates and solvates thereof from about 0.1% w/w to about 99% w/w of the composition, maltodextrin from about 0.1% to about 99% w/w of the composition, optionally with one or more pharmaceutically acceptable excipients from about 0.9% to about 97% w/w of the composition, wherein the composition further comprises not more than about 1% w/w of 2-acetamido-2-(4-octanoylphenethyl)-propane-1,3-diyldiacetate and/or not more than about 1% w/w of 2-acetamido-2-(4-octylphenethyl)propane-1,3-diyldiacetate, when stored at a temperature of about 40° C. and relative humidity of about 75% for 3 months or more.

7. The composition according to claim 1, wherein the pharmaceutically acceptable excipient(s) is selected from a group, comprising diluents/fillers, binders, disintegrants, lubricants, glidants, compression aids, colors, sweeteners, preservatives, surfactants, suspending agents, dispersing agents, film formers, flavors, printing inks, used either alone or in combination thereof.

8. The compositions according to claim 6, wherein the ratio of active agent(s) to maltodextrin is from about 0.1:100 to about 100:0.1.

9. A process for the preparation of stable oral pharmaceutical compositions according to claim 1, wherein the process comprises of the following steps:
i) treating fingolimod with atleast one excipient(s) which is not a sugar alcohol,
ii) optionally adding one or more other pharmaceutically acceptable excipients, and
iii) formulating the material of step (i) and (ii) into a suitable dosage form.

10. A process for the preparation of stable oral pharmaceutical compositions according to claim 8, wherein the process comprises of the following steps:
i) treating fingolimod with saccharide(s),
ii) optionally adding one or more pharmaceutically acceptable excipients, and
iii) formulating the material of step (i) and (ii) into a suitable dosage form.

11. A process for the preparation of stable oral pharmaceutical compositions according to claim 9, wherein the process comprises of the following steps:
i) treating fingolimod with maltodextrin,
ii) optionally adding one or more pharmaceutically acceptable excipients, and
iii) compressing the granules, to form a tablet or filling into capsules.

12. A method of using stable pharmaceutical compositions according to claim 1 for treatment of patients with autoimmune disorders particularly with relapsing forms of multiple sclerosis to reduce the frequency of clinical exacerbations and to delay the accumulation of physical disability.

13. A method of treating a patient according to claim 1 suffering from autoimmune disorders particularly with relapsing forms of multiple sclerosis to reduce the frequency of clinical exacerbations and to delay the accumulation of physical disability.
